(19) United States
(12) Patent Application Publication
(10) Pub. No.: US 2024/0341491 A1
(43) Pub. Date: Oct. 17, 2024

(54) ELONGATED BODY CUSHION

(71) Applicant: Purple Innovation, LLC, Lehi, UT (US)

(72) Inventor: Wade Hoover, Lehi, UT (US)

(21) Appl. No.: 18/625,184

(22) Filed: Apr. 2, 2024

Related U.S. Application Data
(63) Continuation-in-part of application No. 29/779,639, filed on Apr. 20, 2021, now Pat. No. D020307.

(51) Int. Cl. A47C 20/02; A61M 21/02

(52) U.S. Cl. CPC A47C 20/02; A61M 21/02

(57) ABSTRACT

A cushion, such as a body cushion, includes a cover that includes a support grid, as well as a primary cushion within the cover. The cushion and, thus, its cover and primary cushion may be elongated. For example the cushion, its cover, and its primary cushion may have cylindrical shapes. Methods of manufacturing and using such cushions are also disclosed.

### CROSS-REFERENCE TO RELATED APPLICATIONS

This application is a continuation-in-part of U.S. application Ser. No. 29/779,639, filed on Apr. 20, 2021 and titled CYLINDRICAL CUSHION (“the 639 Design application”), issued as U.S. Pat. No. D1,020,307 on Apr. 2, 2024. The entire disclosure of the “639 Design application is hereby incorporated herein.

This application is related to U.S. application Ser. No. 16/901,783, filed on Jun. 15, 2020 and titled PILLOWS AND COVERS (“the '783 Application”). The entire disclosure of the '783 Application is hereby incorporated herein.

### TECHNICAL FIELD

This disclosure relates generally to cushions, such as body cushions, and, more specifically, to cushions with covers that include support grids. More specifically, this disclosure relates to elongated body cushions, including but not limited to cylindrical body cushions, with covers that include support grids. Methods of manufacturing and using such cushions are also disclosed.

### SUMMARY

A cushion of this disclosure includes a cover and a primary cushion. The cover includes a support grid. The cushion may comprise a pillow (e.g., a body pillow, a neck roll pillow, a lumbar pillow or back pillow, a knee pillow, an orthopedic pillow, a sleeping pillow, etc.) or any other type of cushion.

The cover of the cushion includes an outer fabric layer and a support grid. The outer fabric layer includes an outer surface and an inner surface. The outer surface defines a major surface of an exterior of the cover. The inner surface defines at least a portion of an interior of the cover. The support grid is positioned adjacent to the outer fabric layer. For example, the support grid may be positioned adjacent to the inner surface of the fabric layer, within the interior of the cover. Alternatively, the support grid may be positioned over the outer surface of the cover.

The support grid may be substantially coextensive with the outer fabric layer (e.g., with the inner surface of the outer fabric layer, with the outer surface of the outer fabric layer, etc.). Thus, the support grid may extend beneath or over substantially all of the major surface of the cover. The support grid may be relatively thin and, thus, comprise a support web. A thickness of the support grid may be substantially uniform, or some portions of the support grid (e.g., central portions, etc.) may be thicker than other portions of the support grid (e.g., edge portions, etc.).

Optionally, the cover may include an inner fabric layer. The inner fabric layer may be located within the interior of the cover and superimposed with the outer fabric layer. In embodiments where the support grid is located adjacent to the inner surface of the outer fabric layer, the inner fabric layer may be located within the support grid, on an opposite side of the support grid from the outer fabric layer.

As another option, the cover may include peripheral fabric layer. The peripheral fabric layer may be joined to edges of the outer fabric layer. Depending on a shape of the cover, the peripheral fabric layer may define ends and/or a periphery (e.g., a gusset, etc.) of the cover.

The innermost layer of the outer fabric layer, the support grid, and the inner fabric layer (if any) may define a receptacle of the cover. The receptacle of the cover may receive a primary cushion. The primary cushion may include any type of cushion suitable for an intended purpose of the cushion. Without limitation, in embodiments where the cushion comprises a pillow, the primary cushion may comprise a foam cushion, a gel cushion, a loose fill cushion, a fiberfill cushion, or any other cushion suitable for use as a pillow.

Method of manufacturing and using cushions that includes covers with support grids are also disclosed.

Other aspects of the disclosed subject matter, as well as features and advantages of various aspects of the disclosed subject matter, should become apparent to those of ordinary skill in the art through consideration of the ensuing description, the accompanying drawings, and the appended claims.

### BRIEF DESCRIPTION OF THE DRAWINGS

In the drawings:

FIG. 1 is a perspective view of an embodiment of a cushion of this disclosure, which includes a cushion cover with a support grid and a primary cushion within the cushion cover;

FIG. 2 is a front view of the cushion of FIG. 1;

FIG. 3 is a first end view of the cushion of FIG. 1;

FIG. 4 is a second end view of the cushion of FIG. 1;

FIG. 5 is a cross-sectional representation of the cushion of FIG. 1;

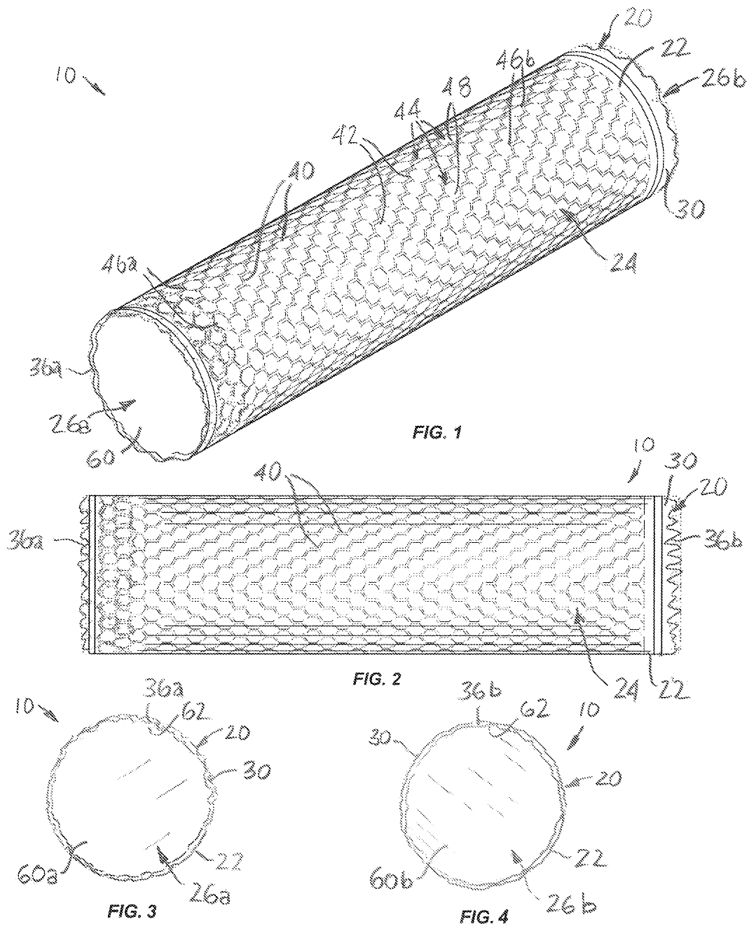

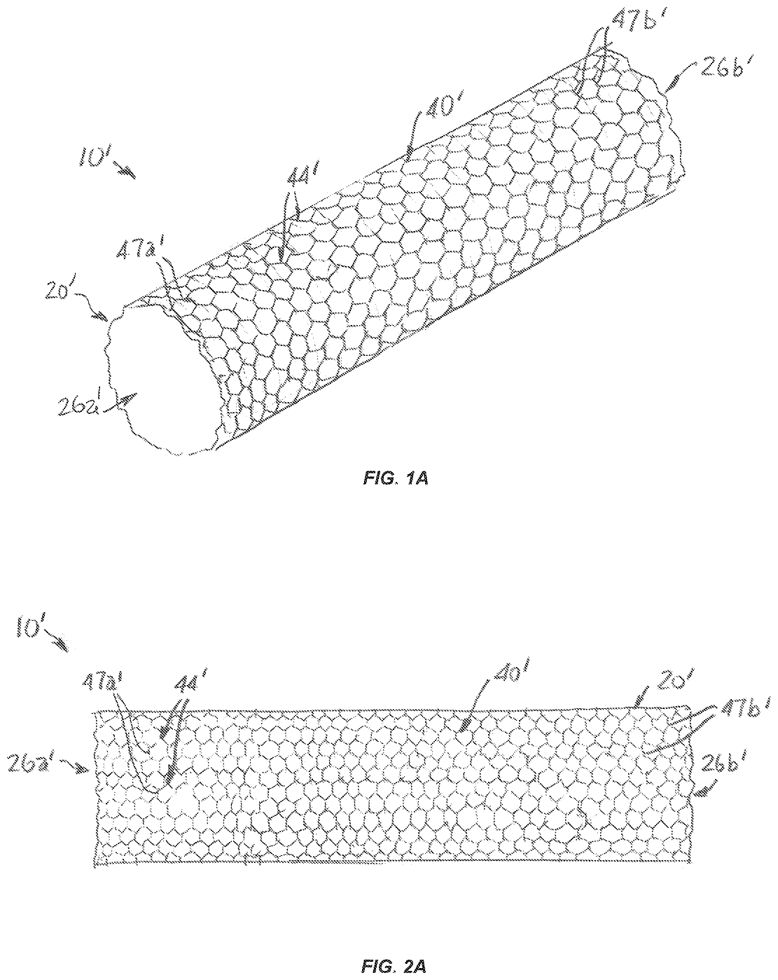

FIG. 1A is a perspective view of another embodiment of a cushion of this disclosure;

FIG. 2A is a front view of the cushion of FIG. 1A.

FIG. 6 is a cross-sectional representation of another embodiment of a cushion of this disclosure;

FIG. 7 is a perspective view of yet another embodiment of a cushion of this disclosure;

FIG. 8 is a cross-sectional representation of the cushion of FIG. 7; and

FIGS. 9-13 are cross-sectional representations showing various embodiments of primary cushions of a cushion of this disclosure.

### DETAILED DESCRIPTION

With reference to FIGS. 1-5, an embodiment of a cushion 10 is depicted. The cushion 10 includes a cover 20 and a primary cushion 70 within the cover 20.

The cover 20 has an outer surface 22. A majority of the outer surface 22 or all of the outer surface 22 may comprise a major surface 24 of the cover 20. The outer surface 22 of the cover 20 may also include one or more peripheral surfaces 26. In the depicted embodiment, in which the cover 20 has a substantially cylindrical shape or a cylindrical shape, the major surface 24 may comprise the curved portion of the outer surface 22 of the cover 20, while the peripheral surfaces 26 may comprise ends 26a and 26b of the cover 20. The cover 20 also includes an interior 28, or a primary receptacle, which may receive the primary cushion 70.

The cover 20 of the cushion 10 includes an outer fabric layer 30 and a support grid 40. Optionally, the cover 20 may include an inner fabric layer 50. As another option, the cover 20 may include one or more peripheral fabric layers 60.

The outer fabric layer 30, the optional inner fabric layer 50, and each optional peripheral fabric layer 60 may comprise any suitable material that is pliable and will enable an individual to enjoy the support and cushioning properties of the primary cushion 70, as enhanced by the support grid 40. The outer fabric layer 30 and any peripheral fabric layers 60 may be formed from a fabric that provides a desired feel (e.g., texture, moisture wicking, cooling sensation, etc.) for an individual who uses the cushion. Without limitation, the outer fabric layer 30, the optional inner fabric layer 50, and any optional peripheral fabric layer 60 may comprise, consist essentially of, or consist of natural fibers (e.g., cotton, bamboo viscose, silk, linen, Tencel, etc.) or synthetic fibers (polyester, nylon, lycra, rayon (a “semi-synthetic” fabric), etc.), or they may comprise blends of fibers. In some embodiments the material may exhibit some stretch, which may be provided by way of synthetic fibers in the material, a knit of the fabric (a Jersey knit, etc.), or a combination of fibers and knit.

The outer fabric layer 30 of the cover 20 includes an outer surface 32 and an inner surface 34. The outer surface 32 may define the major surface 24 of the cover 20 and, thus, at least a portion the outer surface 22 of the cover 20. The inner surface 34 may define at least part of the interior 28, or a primary receptacle, of the cover 20. In the embodiment of cover 20 shown in FIGS. 1-5, the outer fabric layer 30 includes side edges 36a and 36b and end edges 38a and 38b. The end edges 38a and 38b may be secured to each other (e.g., with a seam defined by sewing, welding, etc.). The side edges 36a and 36b may at least partially define the ends 26a and 26b of the cover 20.

In the embodiment of cushion 10 illustrated by FIGS. 1-5, the ends 26a and 26b of the cover 20 are further defined by peripheral fabric layers 60a and 60b. Edges 62 of the peripheral fabric layers 60a and 60b are secured to the side edges 36a and 36b, respectively, of the outer fabric layer 30 (e.g., with a seam defined by sewing, welding, etc.). Shapes of the peripheral fabric layers 60a and 60b may define a shape of the cover 20. For example, the peripheral fabric layers 60a and 60b are circular; thus, the cover 20 has a cylindrical shape. As a few other non-limiting examples, the peripheral fabric layers 60a and 60b may have other round shapes (e.g., ovals, ellipses, shapes with rounded corners and curved sides, etc.), convex polygonal shapes (e.g., triangles, rectangles (including squares), pentagons, hexagons, octagons, etc.), star polygonal shapes, and the like.

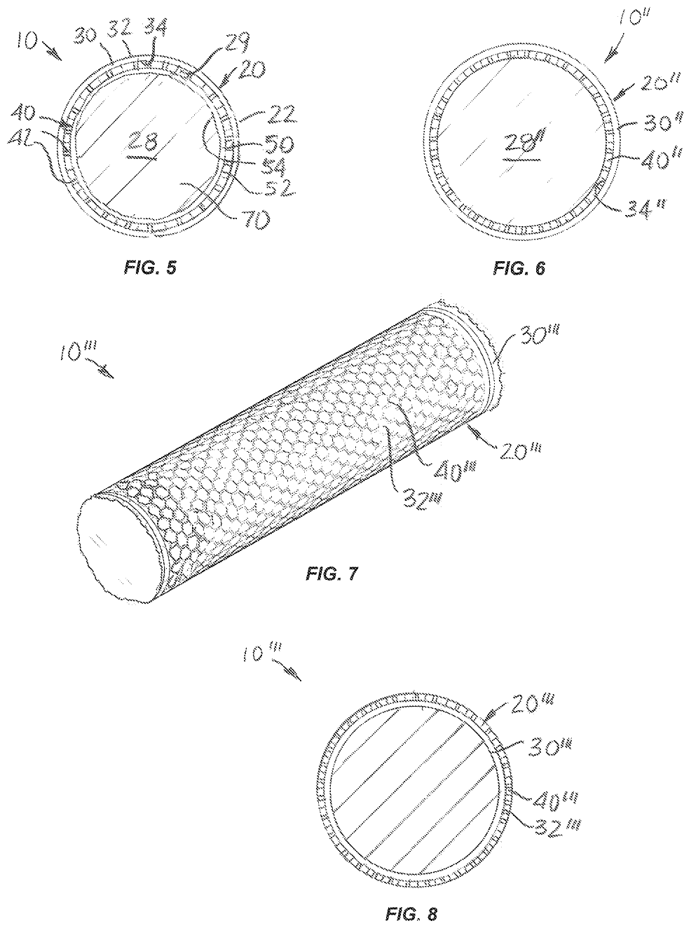

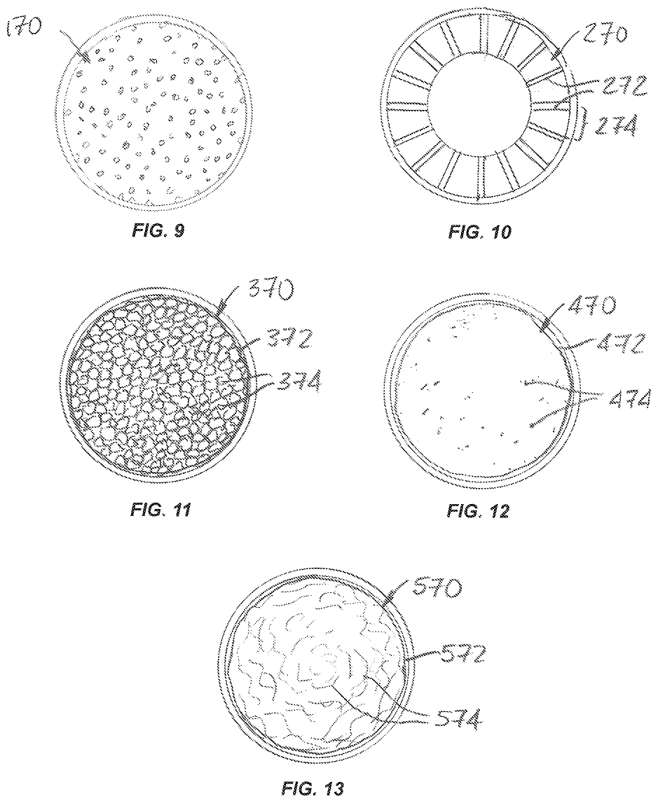

Referring specifically to FIG. 5, the cover 20 may optionally include an inner fabric layer 50. The inner fabric layer 50 may be superimposed over the inner surface 34 of the outer fabric layer 30. More specifically, an outer surface 52 of the inner fabric layer 50 may face, or oppose, the inner surface 34 of the outer fabric layer 30 without being secured to the inner surface 34. Side edges 56a and 56b of the inner fabric layer 50 may be secured to corresponding side edges 36a and 36b of the outer fabric layer 30 (e.g., with a seam defined by sewing, welding, etc.). End edges 58a and 58b of the inner fabric layer 50 may be secured to each other (e.g., with a seam defined by sewing, welding, etc.). An arrangement of the inner fabric layer 50 and the outer fabric layer 30 may define a secondary receptacle 29 of the cover 20. The support grid 40 may reside within the secondary receptacle 29. An inner surface 54 of the inner fabric layer 50 may at least partially define the interior 28, or primary receptacle, of the cover 20.

The support grid 40 may be superimposed with and positioned between the outer fabric layer 30 and the inner fabric layer 50 of the cover 20. The support grid 40 may include a plurality of dividers 42 that define cells 44 of the support grid 40. The divider(s) 42 that define(s) each cell 44 also define a void 48 within the cell 44. In some embodiments, the dividers 42 are interconnected to each other, providing the appearance of intersecting dividers 42.

As illustrated, each cell 44 of the support grid 40 has a hexagonal shape. Thus, the support grid 40 comprises a hexagonal grid. As shown in FIGS. 1 and 2, the support grid 40 is arranged with pointed sides 46a and 46b of the hexagonal cells 44 in general alignment with the ends 26a and 26b of the cover 20. Alternatively, as shown in FIGS. 1A and 2A, the support grid 40′ may be arranged with flat sides 47a′ and 47b′ of the hexagonal cells 44′ in alignment with the ends 26a′ and 26b′ of a cover 20′. Such an arrangement may enable the hexagonal grid to readily collapse during end 26a′-to-end 26′b compression of the cushion 10′ of which the cover 20′ is a part. Support grids 40 with cells 44 that have other shapes (e.g., triangles, squares, diamonds, pentagons, circles, octagons, etc.) or combinations of shapes are also within the scope of this disclosure. U.S. Patent Application No. 63/548,213, the entire disclosure of which is hereby incorporated herein, describes compressible cushions with covers that include collapsible support grids 40.

With returned reference to FIGS. 1, 2, and 5, the support grid 40 may be relatively thin (e.g., it may have a maximum thickness of 1 cm or less, etc.). Such a support grid 40 may be referred to as a “support web.” In embodiments where heights of the dividers 42 of such a support grid 40, which correspond to a thickness of a portion of the support grid 40 in which the dividers 42 are located, do not substantially exceed (e.g., by more than 25%) widths or thicknesses of the dividers 42, the dividers 42 of such a support grid 40 may be referred to as strands or filaments. Due to the relative thinness of such a support grid 40, its cells 44 may not buckle when placed under a load; instead, they may bulge. In embodiments where the heights of the dividers 42 of a support grid 40 exceed the widths or thickness of the dividers 42, the dividers may be referred to as walls.

Alternatively, the support grid 40 may be relatively thick (e.g., its maximum thickness may exceed 1 cm, etc.). Depending upon the height of each divider 42, or wall, relative to a thickness or width of the divider, the cells 44 of such a support grid may buckle when placed under a load. Each cell 44 that buckles when placed under a load may be referred to as a “buckling column.” Some non-limiting examples of such a support grid are described by U.S. Pat. Nos. 5,749,111, 6,026,527, 7,060,213, 7,076,822, and 8,919,750, the entire disclosures of which are hereby incorporated herein.

In some embodiments, a thickness of the support grid 40 may be constant or substantially constant (e.g., it may include curved edges, tapered edges, etc.). In other embodiments, the thickness of the support grid 40 may vary from one location to another; thus, the support grid 40 may be contoured. For example, a thickness of a central region 40c of the support grid 40, which is located midway between the ends 26a and 26b of the cover 20 may exceed a thickness of end regions 40e of the support grid 40, which are located adjacent to the ends 26a and 26b of the cover 20. Such a configuration may enable the support grid 40 to provide additional support or cushioning at locations where an individual is most likely to rest part of their body. As another example, a thickness of the end regions 40e of the support grid 40 may exceed a thickness of the central region 40c of the support grid 40, which may guide a part of an individual's body supported or cushioned by the support grid 40 toward the central region 40c of the support grid 40.

The support grid 40 may comprise a compressible, resilient, elastomeric material. Without limitation, such a material may comprise a thermoplastic elastomer (TPE). The thermoplastic elastomer may comprise a block copolymer (e.g., a triblock copolymer, such as an A-B-A triblock copolymer, etc.). The thermoplastic elastomer may be part of an elastomeric gel. The elastomeric gel may comprise a plasticizer-extended block copolymer. Some non-limiting examples of plasticizer-extended block copolymers are described by U.S. Pat. Nos. 5,994,450, 6,797,765, and 7,964,664, the entire disclosures of which are hereby incorporated herein by reference. These plasticizer-extended block copolymers include plasticizer-extended A-B-A block copolymers, such as oil-extended styrene-[ethylene-(ethylene-propylene)]-styrene (SEEPS) block copolymers, oil-extended styrene-(ethylene-butylene)-styrene (SEBS) block copolymers, and other oil-extended A-B-A block copolymers. Alternatively, the support grid 40 may be formed from other materials, such as rubber (e.g., natural latex, polyurethane, silicone, butyl rubber, etc.), foam rubber (e.g., natural latex, polyurethane, viscoelastic foams, etc.), or any other suitable material.

In embodiments where the cover includes a secondary receptacle 29, the secondary receptacle 29 may hold the support grid 40 in place adjacent to the outer fabric layer 30 or, more specifically, adjacent to the inner surface 34 of the outer fabric layer 30. Alternatively, the support grid 40 may be secured to the inner surface 34 of the outer fabric layer 30. For example, the support grid 40 may be formed on the inner surface 34 of the outer fabric layer 30 (e.g., by a molding process, etc.). As another example, the support grid 40 may be formed separately from the outer fabric layer 30 and then adhered to the inner surface 34 of the outer fabric layer 30 (e.g., by melt bonding, with an adhesive material, etc.). As another alternative, the support grid 40 may be formed on or secured to the outer surface 52 of the inner fabric layer 50. As yet another alternative, the support grid 40 may be secured to both the inner surface 34 of the outer fabric layer 30 and the outer surface 52 of the inner fabric layer 50, for example, by forming the support grid 40 on one of these surfaces and adhering it to the other of these surfaces or by adhering the support grid 40 top both of these surfaces. When the support grid 40 is formed on or melt bonded to the outer fabric layer 30 and/or the inner fabric layer 50, a material of the support grid 40 may impregnate the outer fabric layer 30 and/or inner fabric layer 50 to mechanically couple the support grid 40 to the inner surface 34 of the outer fabric layer 30 and/or to the outer surface 52 of the inner fabric layer 50.

Turning now to FIG. 6, another embodiment of a cushion 10″ is depicted. The cushion 10″ includes a cover 20″ that includes an outer fabric layer 30″ and a support grid 40″, but no inner fabric layer. The outer fabric layer 30″ may be configured in the same manner as or in a similar manner to the outer fabric layer 30 described in reference to FIGS. 1-5. The support grid 40″ may be configured in the same manner as or in a similar manner to the support grid 40 described in reference to FIGS. 1-5. More specifically, the support grid 40″ may be superimposed with the outer fabric layer 30″. The support grid 40″ may be optionally secured to the inner surface 34″ of the outer fabric layer 30″ (e.g., by being formed on the inner surface 34″ of the outer fabric layer 30″, by being adhered to the inner surface 34″ of the outer fabric layer 30″, etc.). In such an embodiment, the support grid 40″ may at least partially define an interior 28″, or a primary receptacle, of the cover 20″.

FIGS. 7 and 8 illustrate another embodiment of a cushion 10′″. The cushion 10′″ includes a cover 20′″ with an outer fabric layer 30′″ and a support grid 40′″ over an outer surface 32′″ of the outer fabric layer 30′″. Despite the difference in the arrangement of the outer fabric layer 30′″ and the support grid 40′″ from the arrangement of the outer fabric layer 30 and the support grid 40 of the embodiment of cushion 10 shown in and described with reference to FIGS. 1-5, the outer fabric layer 30′″ and support grid 40′″ may be the same as or similar to the outer fabric layer 30 and support grid 40′″, respectively, of the embodiment of the cushion 10 shown in and described with reference to FIGS. 1-5. Additionally, the support grid 40′″ may have a columnar (e.g., cylindrical, etc.) shape, which may enable the support grid 40′″ to receive the outer fabric layer 30′″. Such a support grid 40′″ may be formed by securing end edges of a flat support grid to each other. Optionally, the support grid 40′″ may be secured to (e.g., formed on, adhered to, etc.) the outer surface 32′″ of the outer fabric layer 30′″.

Referring generally to FIGS. 5, 6, and 8, the primary cushion 70 of a cushion 10, 10″, 10′″ of this disclosure may be placed and reside within the interior 28, 28″, 28′″, or primary receptacle, of the cover 20, 20″, 20′″ of the cushion 10, 10″, 10′″. The primary cushion 70 may comprise any cushion imparts the cushion 10, 10″, 10′″ with desired cushioning characteristics. For example, the primary cushion 70 may comprise a foam cushion 170 (FIG. 9), a gel cushion 270 (FIG. 10), a loose fill cushion 370, 470 (FIGS. 11 and 12, respectfully), or a fiberfill cushion 570 (FIG. 13). With reference to FIG. 9. some non-limiting examples of foam cushions 170 include latex cushions (e.g., latex, Dunlop latex, Talalay latex, etc.), memory foam cushions, and foam rubber cushions. With reference to FIG. 10, some non-limiting examples of gel cushions 270 include those described by U.S. Pat. Nos. 5,749,111, 6,026,527, 7,060,213, 11,812,880, and 11,844,451, the entire disclosures of which are hereby incorporated herein; thus, a gel cushion 270 may include a plurality of deformable, resilient walls 272 that define an array of buckling columns 274. With reference to FIG. 11, some non-limiting examples of loose fill cushions 370 include a pliable cover 372 that contains pieces of a shredded material 374, such as a foam (see, e.g., U.S. Pat. No. 7,461,424, the entire disclosure of which is hereby incorporated herein) or a gel (e.g., a gel elastomer, etc.) (alone or in combination with shredded foam). With reference to FIG. 12, some other non-limiting examples of loose fill cushions 470 include a pliable cover 472 that contains a fill material 474 that comprises feathers, down, synthetic down, buckwheat shells, microbeads, or the like. Loose fill cushions that include any useful combinations of any of the foregoing fill materials 374, 474 (see FIGS. 11 and 12) are also within the scope of this disclosure. With reference to FIG. 13, some non-limiting examples of fiberfill cushions 570 include a pliable cover 572 that contains fibers 574 formed from cotton, polyester, or any other material that, when formed into fibers, may be used to cushion a part of an individual's body.

A cushion 10, 10′, 10″, 10′″ of this disclosure may comprise a body, knee, and back pillow, as depicted. The cushion 10, 10′, 10″, 10′″ may alternatively comprise a neck pillow, a lumbar pillow or a back pillow, a knee pillow, an orthopedic pillow (e.g., with a contoured primary cushion 70), a sleeping pillow, or any other type of cushion.

Methods of making a cover 20, 20′, 20″, 20′″ of this disclosure should be apparent from the foregoing.

In use, a cushion 10, 10′, 10″, 10′″ of this disclosure may support a part of an individual's body. For example, the cushion 10, 10′, 10″, 10′″ may be positioned behind the individual's back, between the individual's knees, beneath the individual's knees, under the individual's neck, under the individual's head, etc. The primary cushion 70 of the cushion 10, 10′, 10″, 10′″ may provide the majority of the cushioning for the body part.

The support grid 40, which extends over the outside of the primary cushion 70, may supplement or even enhance the cushioning provided by the primary cushion 70. For example, the dividers 42 of the support grid 40 and the voids 46 in the cells 44 of the support grid 40 may ensure that some space remains between the body part and the primary cushion 70, which may enable heat to flow away from the body part as the cushion 10, 10′, 10″, 10′″ supports it, which may provide a cooling effect that may have a positive effect on inflammation of the body part (it may prevent further inflammation, reduce inflammation, etc.).

As another example, as the body part sinks into the primary cushion, the support grid 40 may have a hammocking effect that hugs or cradles the body part, which may induce deep pressure stimulation that may lead to increased levels of serotonin (a mind-boosting hormone) and melatonin (a hormone that induces sleep) and/or reduce cortisol (a stress hormone), providing the individual with a sense of security and, thus, have a calming effect on the individual, enable the individual to fall asleep more quickly, and improve the individual's sleep quality.

Features from different embodiments of the disclosed subject matter may be employed in combination.

Although the disclosure provides many specifics, the specifics should not be construed as limiting the scope of any of the claims, but merely as providing illustrations of some embodiments of elements and features of the disclosed subject matter that fall within the scopes of the claims. Other embodiments of the disclosed subject matter may be devised that are also within the scopes of the claims. Accordingly, the scope of each claim is limited only by its plain language and the legal equivalents thereto.

What is claimed:

1. An elongated body cushion, comprising:
a cover including:
an outer fabric layer including an inner surface defining an interior of the cover and an outer surface defining major surfaces of an exterior of the cover;
a support grid adjacent to the inner surface of the fabric layer and extending across substantially an entirety of the inner surface; and
a primary cushion within the interior of the cover.

2. The elongated body cushion of claim 1, wherein the cover further includes:
peripheral fabric layer joined to edges of the outer fabric layer and defining ends and/or a periphery of the cover.

3. The elongated body cushion of claim 2, wherein:
the outer fabric layer has a cylindrical shape; and
the peripheral fabric layer is located at ends of the cylindrical shape.

4. The elongated body cushion of claim 3, wherein the support grid comprises a hexagonal grid.

5. The elongated body cushion of claim 4, wherein the support grid comprises a support web.

6. The elongated body cushion of claim 3, wherein a central thickness of the support web at locations between the ends of the cylindrical shape exceeds an end thickness of the support web at locations adjacent to the ends of the cylindrical shape.

7. The elongated body cushion of claim 1, wherein the cover further includes:
an inner fabric layer within the interior of the cover, on an opposite side of the support grid from the outer fabric layer, and defining a receptacle of the cover that receives the primary cushion.

8. The elongated body cushion of claim 7, wherein a material of the support grid impregnates the inner fabric layer.

9. The elongated body cushion of claim 1, wherein a material of the support grid impregnates the outer fabric layer.

10. The elongated body cushion of claim 1, wherein the support grid comprises a hexagonal grid.

11. The elongated body cushion of claim 10, wherein flat sides of hexagons of the hexagonal grid are aligned with sides of the elongated body cushion.

12. The elongated body cushion of claim 1, wherein the support grid comprises a support web.

13. The elongated body cushion of claim 1, wherein the primary cushion comprises one of a foam cushion, a gel cushion, a loose fill cushion, and a fiberfill cushion.

14. The elongated body cushion of claim 13, wherein the primary cushion is a foam cushion and comprises a latex pillow or a memory foam pillow.

15. The elongated body cushion of claim 13, wherein the primary cushion is a gel cushion and comprises an array of buckling columns.

16. The elongated body cushion of claim 13, wherein the primary cushion is a loose fill cushion and comprises shredded foam.

17. The elongated body cushion of claim 13, wherein the primary cushion is a loose fill cushion and comprises shredded gel.

18. The elongated body cushion of claim 13, wherein the primary cushion is loose fill cushion and comprises feathers, down, synthetic down, buckwheat shells, or microbeads.

19. The elongated body cushion of claim 13, wherein the primary cushion is a fiberfill cushion and comprises cotton or polyester.

20. The elongated body cushion of claim 1, wherein the primary cushion comprises one of a body pillow, a neck roll pillow, a lumbar pillow or back pillow, a knee pillow, an orthopedic pillow, and a sleeping pillow.
